(19) United States
(12) Patent Application Publication
(10) Pub. No.: US 2004/0204443 A1
(43) Pub. Date: Oct. 14, 2004

(54) Method for inducing pain relief using imidazo[1,2-a]pyridine derivatives

(76) Inventor: Arthur Zaks, Fort Lee, NJ (US)

Correspondence Address: Allan J. Grant, Esq., c/o Carella, Byne, Bain Gilfillan,, Cecchi, Stewart & Olstein, 6 Becker Farm Road, Roseland, NJ, 07068, US

(21) Appl. No.: 10/764,857

(22) Filed: Jan. 26, 2004

Related U.S. Application Data
(60) Provisional application No. 60/442,827, filed on Jan. 27, 2003.

(51) Int. Cl. A61K 31/4745

(52) U.S. Cl. 514/303

(57) ABSTRACT

Methods for inducing pain relief, especially chronic pain, in a patient by oral or topical administration of imidazo[1,2-a]pyridine derivatives, including zolpidem, are disclosed along with dosages and formulations of the active ingredient.

This application claims priority of U.S. Provisional Application Serial No. 60/442,827, filed 27 Jan. 2003, the disclosure of which is hereby incorporated by reference in its entirety.

### FIELD OF THE INVENTION

The present invention relates to use of pharmaceutical compositions imidazo [1,2-a]pyridine derivatives in treating pain and related disorders wherein said derivatives may be administered orally, topically or by other routes.

### BACKGROUND OF THE INVENTION

Imidazo [1,2-a]pyridines are known in the field of treatment of neurological diseases and conditions, especially those related to hypnotic and convulsant symptoms. Some of the more useful derivatives have been patented (see, for example, Kaplan et al., U.S. Pat. No. 4,382,938, issued 10 May 1983). The structure and activity of this class of compounds was described even earlier. (See, for example, Almirante et al., I, J. Med. Chem., vol. 8, (1965) pp. 305-312; Almirante et al., II, J. Med. Chem., vol. 12, (1969) pp.123-126).

These compounds have been shown to possess anxiolytic, anti-anoxic, sleep-inducing, hypnotic and anticonvulsant properties and have been found useful for the treatment of anxiety states, sleep disorders and other neurological and psychiatric complaints. They have also been found efficacious in the treatment of vigilance disorders, such as behavioral disorders attributable to cerebral vascular damage and to the cerebral sclerosis encountered in geriatrics, as well as in the treatment of epileptic vertigo (for example, from cranial traumatisms) and the treatment of metabolic encephalopathies.

Many of the compounds described previously have exhibited strong binding to central and peripheral benzodiazepine receptors (see for example Anzini, M. et al., J. Med. Chem. 39 4275-4284 (1996) and Trapani, G. et al. J. Med. Chem. 40:3109-3118 (1997)). The 2-(iodophenyl)-imidazo[1.2-a]pyridines exhibit strong binding to peripheral benzodiazepine receptors with much weaker binding to central benzodiazepine receptors and are useful for imaging. The peripheral benzodiazepine receptors are modulated by hormones and drugs and reflect the effects of emotional stress, and hypertension. (See, for example, Katsifis et al, U.S. Pat. No. 6,379,649, issued 30 Apr. 2002)

A number of 2-aryl substituted imidazo[1,2-a]pyridines having anxiolytic, hypnotic, anticonvulsant, analgesic and other properties have been reported (Almirante L. et al., supra; Langer S. Z. et al., Pharmacol. Biochem, Behav. 29 763-766 (1988); Bourguignon, J-J., “Endogenous and Synthetic Ligands of Mitochondrial Benzodiazepine Receptors: Structure Affinity Relationships” in Giesen-Grouse. E. ed. Peripheral Benzodiazapine Receptors, Academic Press, London (1993)).

In addition, animal studies have shown some derivatives to be useful in the treatment of a variety of neurological problems, including action as analgesics, anti-inflammatories, and antipyretics. These derivatives have also been shown to have hypothermal activity and to act as anticonvulsants. See Almirante et al (1969), supra.

Among the more commercially successful of such derivatives has been the pharmaceutically useful compound known as AMBIEN® (or zolpidem tartrate, N,N,6-trimethyl-2-p-tolyl-imidazo[1,2-a]pyridine-3-acetamide (commonly formulated as the L-(+)-tartrate, 2:1, which may also contain a number of inactive ingredients). This derivative is currently prescribed for short term treatment of insomnia and is commonly administered orally. It has been shown to decrease sleep latency and to increase the duration of sleep. No literature has been found showing that this compound has any analgesic or pain relieving effect and it is not currently recognized as useful for such. For example, the Physician's Desk Reference (1999) does not list this compound for use as an analgesic but rather for treatment of insomnia. Even where some analgetic effects have been noted (see, for example, Almirante et al (1969), these were selected derivatives tested with laboratory pain testing procedures and did not include amelioration of the chronic pain or pain due to cancer reported for the methods of the present invention.

### BRIEF DESCRIPTION OF THE INVENTION

The present invention relates to a method for inducing pain relief in a mammal, preferably a human patient, comprising administering to a mammal afflicted with pain and in need of analgesia, or pain relief, an effective pain-relieving amount of a composition comprising an imidazo[1,2-a]pyridine in a pharmaceutically acceptable carrier. A stimulant may be administered contemporaneously with said composition, or as part of it.

In a preferred embodiment of such method, said imidazo[1,2-a]pyridine is N,N,6-trimethyl-2-p-tolyl-imidazo[1,2-a]pyridine-3-acetamide, most preferably formulated as the tartrate salt, zolpidem tartrate. In an additional preferred embodiment, said imidazo[1,2-a]pyridine composition is administered orally, topically or by other routes.

### DETAILED DESCRIPTION OF THE INVENTION

In one aspect, the present invention relates to a method for inducing analgesia, or pain relief, in a mammal, including a human patient, comprising administering to said mammal, including a human patient. in need of pain relief an effective pain-relieving amount of a composition comprising an imidazo[1,2-a]pyridine in a pharmaceutically acceptable carrier. In a preferred embodiment, such composition may also comprise a stimulant, such as ritilin and/or provigil. Such stimulants may also be administered separate from the pain-relieving composition or as part of it and may also be administered before or after said analgesic composition, although preferably contemporaneously therewith.

In a preferred embodiment, the present invention relates to the foregoing method wherein said imidazo[1,2-a]pyridine is N,N,6-trimethyl-2-p-tolyl-imidazo[1,2-a]pyridine-3-acetamide, most preferably wherein said imidazo[1,2-a]pyridine is zolpidem tartrate.

In another preferred embodiment of these methods, the imidazo[1,2-a]pyridine composition is administered orally. In a further preferred embodiment, the imidazo[1,2-a]pyridine is administered topically.

Imidazo[1,2-a]pyridines have the following general formula:

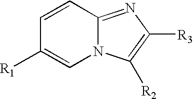

Active derivatives useful in the methods of the invention include those where R₁=alkyl, R₂=methylamino and R₃=alkylphenyl.

N,N,6-trimethyl-2-p-tolyl-imidazo[1,2-a]pyridine-3-acetamide is a structure wherein R₁=methyl, R₂═—CH₂CON(CH₃)₂ and R₃=4-methylphenyl and is well known in the art (see, for example, Physician's Desk Reference, current edition). It has the structure:

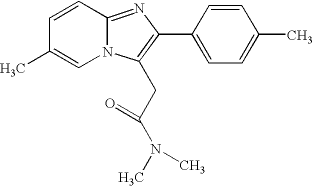

While use of this compound is a highly preferred embodiment, other derivatives of the imidazo[1,2-a]pyridine core structure may be useful in the methods of the invention. These compounds are commonly formulated for use as a salt, for example, tartrate salts wherein usually 2 molecules are bound to tartaric acid. However, the compounds useful in the methods of the invention do not include structures wherein R₁ is hydrogen, or wherein the pyridinyl ring portion comprises a 5-methyl or 7-methyl substituent, or wherein R₂ is hydrogen, methyl, p-chlorophenyl, or p-methylsulfonylphenyl, or wherein R₃ is cyano (CN), cyanomethyl (CNCH₂—), amide (—CONH₂) amidomethyl (—CH₂CONH₂), carboxyl (—COOH), or acetyl (—CH₂COOH), or combinations of these.

In another aspect, the present invention relates to a method for inducing analgesia in a mammal, preferably a human patient, comprising administering to a mammal in need of analgesia an effective analgesia-inducing amount of a composition comprising an imidazo[1,2-a]pyridine in a pharmaceutically acceptable carrier wherein said administration is topical.

In preferred embodiments of all of the methods of the invention, the pain to be treated or ameliorated is chronic pain, such as pain of the extremities and/or joints, as well as pain due to other causes, such as cancer, especially metastatic cancer.

In a further aspect, the present invention relates to a method for inducing pain relief in a mammal comprising administering to a mammal in need of analgesia an effective analgesia-inducing amount of a composition comprising N,N,6-trimethyl-2-p-tolyl-imidazo[1,2-a]pyridine-3-acetamide in a pharmaceutically acceptable carrier. In a preferred embodiment thereof, said administration is oral. It is commonly formulated with L-(+)-tartrate (2:1).

The present invention also relates to a method for treating and/or preventing pain in a mammal comprising administering to a mammal in need of analgesia an effective analgesia-inducing amount of a composition comprising zolpidem, such as zolpidem tartrate, in a pharmaceutically acceptable carrier. In a preferred embodiment thereof, said administration is oral.

The methods of the invention commonly involve administration of compositions comprising the analgesic agents disclosed herein. These compositions may be administered by any means effective to induce analgesia, preferably orally or topically.

A pharmaceutical composition in accordance with the invention may be administered orally, topically, parenterally, including intravenously, rectally or nasally.

For oral administration, the pharmaceutical composition, such as a composition containing zolpidem, may be in the form of tablets, pills, lozenges, capsules, powders, granules, suspensions, emulsions, and tinctures. Time release methods of administration, such as time release capsules, may also be used without lessening the effect of the invention. These can include both coated granules and multi-layer tablets.

Solid forms for administration may contain pharmaceutically acceptable inert materials, such as binders (for example, gum acacia, gelatin, corn starch, gum tragacanth, sodium alginate, carboxymethylcellulose or polyethylene glycol), sweeteners (such as sucrose, lactose, glucose, aspartame or saccharine), flavoring agents (e.g., peppermint oil, oil of wintergreen, cherry, orange or raspberry flavorings), coating agents (which may be any suitable polymeric substance, including, but not limited to polymers or copolymers of acrylic acid and/or methacrylic acid and/or their esters, waxes, fatty alcohols, zein, shellac or gluten), preservatives (sodium benzoale, vitamin E, alpha-tocopherol, ascorbic acid, methyl paraben, propyl paraben or sodium bisulphite), lubricants (for example, magnesium stearate, stearic acid, sodium oleate, sodium chloride or talc) and other diluents or excipients (including lactose, sorbitol, mannitol, dextrose, kaolin, cellulose, calcium carbonate, calcium silicate or dicalcium phosphate).

Methods well known in the art for making formulations are found in, for example, Remington: The Science and Practice of Pharmacy, (19th ed.) ed. A. R. Gennaro A R., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel.

If a liquid form is used, a suitable carrier (i.e., a liquid carrier) will commonly be included (in addition to the above-recited additives) and these may include various diluents and excipients water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof. liquid forms may be administered orally or be in the form of an emulsion suitable for inhalation (for example, in the form of a nasal spray), in which case an inhalable propellant (with low toxicity) may be employed. Common propellants included carbon dioxide or nitrous oxide.

Suspensions for oral administration may also contain dispersing agents (such as lecithin, polyoxyethylene esters of fatty acids such as stearic acid, polyoxyethylene sorbitol mono- or di-oleate, stearate, laurate, polyoxyethylene sorbitan mono- or di-oleate, stearate, laurate and the like) and/or suspending agents (including methylcellulose, hydroxypropylmethylcellulose, polyvinylpyrrolidone, sodium carboxyethylcellulose, sodium alginate or cetyl alcohol. In addition, natural gums such as gum acacia or gum tragacanth may be included as emulsifying agents where oral administration is contemplated.

In a highly preferred embodiment, the methods of the invention specifically encompass topical administration. As such, the pharmaceutical compositions useful in the methods of the invention may be in any form suitable for topical application, especially as a cream, ointment, gel, jelly, tincture, suspension or emulsion. The pharmaceutically acceptable binders, diluents, preservatives, and other excipients recited herein may be included in such formulations.

For other routes of administration, such as rectal, the compositions may be in the form of a suppository. Here, the active ingredient is combined with a suitable non-abrasive and non-irritating agent that, while solid at ordinary temperatures, will melt in the rectum. Suitable agents include cocoa butter and polyethylene glycols.

The dosage forms useful in the methods of the invention will comprise the active ingredient (for example, zolpidem) in a concentration of from 0.01% to 99% by weight, preferably from 0.1% to 10% by weight of the active material. In various embodiments of the methods of the invention, dosages will be from 0.01 mg to 10 mg per kg of body weight, preferably between 0.1 to 5 mg per kg body weight. Of course, dosages and treatment regimens may vary depending on the route of administration and the specific dosage and regimen to be administered will depend on the nature of the malady (i.e., the type and source of pain) and on the inclinations and discretion of the clinician supervising the treatment.

Treatment regimens may also differ depending on whether the administration is part of a course of clinical treatment or is being conducted for purposes of research. Treatment may also vary depending on whether the recipient is a human patient or some other mammal. In addition, the course of treatment may also vary in that treatment may comprise one or more successive administrations of the same or different dosage and may comprise a regular course of treatment at specific intervals or intermittent administration. The methods of the invention may also be used to treat active pain or to prevent anticipated pain resulting from either a disease condition, such as cancer, or from treatment of a malady wherein said treatment is expected to result in pain. Thus, the methods of the invention are equally useful when conducted before or after onset of pain.

### EXAMPLE

A 59 year old man presented with intense, bilateral pain in both feet. The plantar fascia of both feet had been stretched by improperly made orthotics.

The predominant pain was sharp and stinging in nature and was experienced primarily under the heads of the metatarsals, with pain beginning shortly after the patient attempted to stand on his feet. Additionally, arch pain, heel pain and pain in the cuboid area were also experienced. The latter were dull or throbbing but of lower intensity than the metatarsal pain. A physician involved in the case believed these pains to be caused by the bones pressing against the skin and other structures because of the observed ligamentous laxity.

The patient was subsequently treated with Parmelar (nortryptiline), Elavil (amitryptiline), Topamax, Neurontin, Lamictal, Ultram, and Vicodin, with only the latter showing any decrease in pain, although this was considered negligible. Additionally, the patient was treated with topical lidocaine and with a tens machine.

The patient was subsequently given AMBIEN® (zolpidem tartrate, as a narcotic to induce sleep) at 10 mg H.S. The patient subsequently reported a noticeable abatement of pain and was able to walk prior to retiring. Subsequent examination showed that pain abatement lasted for a period of 12 to 14 hours although AMBIEN® has been reported to be metabolized in about 6 to 8 hours. The patient's obesity may have accounted for the long duration of the pain amelioration, since AMBIEN® is lipophilic and may be eliminated more slowly in obese individuals. Effective dosage was about 0.08 mg per kg. In addition, all of the other foot pains reported by the patient were ameliorated by AMBIEN® treatment.

In a follow-up treatment, 5 mg of AMBIEN® was administered. Because AMBIEN® is a soporific, the stimulants ritilin and provigil were co-administered with the AMBIEN®. Administration of such stimulants was restricted due to a condition of angina in said patient so that the full measure of stimulation could not be achieved.

AMBIEN® was also applied topically (in a cream) to this patient with reported pain relief.

A second patient with metastatic cancer was also experiencing pain and had reported little or no relief from common analgesics. AMBIEN® was administered to this patient with subsequent marked analgesic effect.

What is claimed is:

1. A method for inducing pain relief in a mammal comprising administering to a mammal in need of pain relief a pain-relieving amount of a composition comprising an imidazo[1,2-a]pyridine in a pharmaceutically acceptable carrier.

2. The method of claim 1 wherein said imidazo[1,2-a]pyridine is N,N,6-trimethyl-2-p-tolyl-imidazo[1,2-a]pyridine-3-acetamide.

3. The method of claim 1 wherein said imidazo[1,2-a]pyridine is zolpidem tartrate.

4. The method of claim 1 wherein said imidazo[1,2-a]pyridine composition is administered orally.

5. The method of claim 1 wherein said imidazo[1,2-a]pyridine is administered topically.

6. The method of claim 1 wherein said pain is chronic pain.

7. The method of claim 1 wherein said pain is caused by cancer.

8. The method of claim 7 wherein said cancer is a metastatic cancer.

9. The method of claim 1 wherein said composition further comprises a stimulant.

10. The method of claim 9 wherein said stimulant is a member selected from the group consisting of ritilin and provigil.

11. The method of claim 1 wherein said mammal is a human patient.
